(19) United States
(12) Patent Application Publication
(10) Pub. No.: US 2025/0288373 A1
(43) Pub. Date: Sep. 18, 2025

(54) SYSTEM AND METHOD FOR OBTAINING KINEMATICS OF A ROBOTIC CART IN A MULTI-ARM ROBOTIC SURGICAL SYSTEM

(71) Applicant: Sudhir Prem SRIVASTAVA, Gurugram, Haryana (IN)

(72) Inventors: Sudhir Prem SRIVASTAVA, Gurugram (IN); Vishwajyoti Pascual SRIVASTAVA, Gurugram (IN); Rama Krishna Reddy DYAVA, Gurugram (IN); Suraj DWIVEDI, Gurugram (IN); Anson PAUL, Gurugram (IN)

(73) Assignee: Sudhir Prem SRIVASTAVA, Gurugram, Haryana (IN)

(21) Appl. No.: 18/689,839

(22) PCT Filed: Dec. 1, 2023
(86) PCT No.: PCT/IN2023/051124

§ 371 Date: Mar. 6, 2024

(30) Foreign Application Priority Data
Dec. 7, 2022 (IN) 202211070574

(51) Int. Cl. A61B 34/30; A61B 1/00; A61B 34/20

(52) U.S. Cl. CPC A61B 34/30; A61B 1/00149; A61B 2034/2055; A61B 2034/2059

(57) ABSTRACT

A multi-arm robotic surgical system and a method for obtaining kinematics of a plurality of robotic carts with respect to a frame of reference. The system comprises of a laser module and a transmitter coupled to each of the plurality of robotic carts. The laser module is configured to generate a laser line associated with each of the plurality of robotic carts. The transmitter is configured to encode values of angles between the generated laser line associated with each of the plurality of robotic carts and the frame of reference. A master controller is operatively coupled to the plurality of robotic carts and configured to calculate the kinematics of the plurality of robotic carts with respect to the frame of reference, based on the encoded values of the angles.

### TECHNICAL FIELD

The present disclosure generally relates to a multi-arm robotic surgical system for minimally invasive surgery, and more particularly, the disclosure relates to a system and method for obtaining kinematics of a robotic cart in a multi-arm robotic surgical system.

### BACKGROUND

This section is intended to introduce the reader to various aspects of art that may be related to various aspects of the present disclosure, which are described below. This disclosure is believed to be helpful in providing the reader with background information to facilitate a better understanding of the various aspects of the present disclosure. Accordingly, it should be understood that these statements are to be read in this light, and not just as an admissions of prior art.

Robotically assisted surgical systems have been adopted worldwide to replace conventional surgical procedures to reduce number of extraneous tissue(s) that may be damaged during surgical or diagnostic procedures, thereby reducing patient recovery time, patient discomfort, prolonged hospital tenure, and particularly deleterious side effects. In robotically assisted surgeries, the surgeon typically operates a hand controller/master controller/surgeon input device at a surgeon console to seamlessly capture and transfer complex actions performed by the surgeon giving the perception that the surgeon is directly articulating surgical tools/surgical instruments to perform the surgery. The surgeon operating on the surgeon console may be located at a distance from a surgical site or may be located within an operating theatre where the patient is being operated.

The robotically assisted surgeries have revolutionized the medical field and are one of the fastest growing sectors in the medical device industry. One of the key areas of robotically assisted surgeries is the development of surgical robots for minimally invasive surgery. Over the last couple of decades, surgical robots have evolved exponentially and have been a major area of innovation in the medical device industry.

In the robotic assisted surgeries and more specifically in a modular robotic system, one of the challenges is to position the surgical carts in optimum location around the operating table. The main challenge with the existing robotically assisted surgical systems is that as the operating room is an indoor environment, a conventional mode of positioning system is not useful. The conventional positioning systems make use of some kinds of beacons which need to be placed in the operating room. This is not desirable due to the restrictions of the medical regulations, and the possibility of a patient being moved between the operating rooms because of emergencies. Further, the size of an operating room may vary. Another challenge is that the existing multi-arm robotic surgical systems being modular, it is very difficult to determine the orientation of the robotic carts with respect to a frame of reference. Further, another challenge is that this poor information of the orientation of the robotic carts with respect to a frame of reference may lead to collision between robotic arms and surgeon hand cannot be mapped with the surgical instrument tip.

In the light of aforementioned challenges, there is a need for a system and method for obtaining kinematics of a robotic cart in a multi-arm robotic surgical system which will solve the above-mentioned problems related to robotic assisted surgeries.

### SUMMARY OF THE DISCLOSURE

Some or all of the above-mentioned problems related to obtaining kinematics of a robotic cart in a multi-arm robotic surgical system are proposed to be addressed by certain embodiments of the present disclosure.

In an aspect, an embodiment of the present disclosure provides a multi-arm robotic surgical system for obtaining kinematics of a plurality of robotic carts with respect to a frame of reference comprising a plurality of robotic arms each mounted on one of a plurality of robotic carts, an endoscopic camera coupled to a robotic arm out of the plurality of robotic arms, a plurality of surgical instruments each detachably coupled to a distal end of a robotic arm out of the remaining robotic arms, an operating table, and a patient lying on the operating table, whereby the plurality of robotic carts are arranged along the operating table, the system comprising: a laser module provided with each of the plurality of robotic carts, the laser module configured to generate a laser line associated with each of the plurality of robotic carts; a transmitter coupled to each of the plurality of robotic carts, the transmitter configured to encode values of angles between the generated laser line associated with each of the plurality of robotic carts and the frame of reference; and a master controller operatively coupled to the plurality of robotic carts, the master controller configured to calculate the kinematics of the plurality of robotic carts with respect to the frame of reference, based on the encoded values of the angles.

In another aspect, an embodiment of the present disclosure provides a method for obtaining kinematics of a plurality of robotic carts with respect to a frame of reference in a multi-arm robotic surgical system comprising a plurality of robotic arms each mounted on one of a plurality of robotic carts, an endoscopic camera coupled to a robotic arm out of the plurality of robotic arms, a plurality of surgical instruments each detachably coupled to a distal end of a robotic arm out of the remaining robotic arms, an operating table, and a patient lying on the operating table, whereby the plurality of robotic carts are arranged along the operating table, the method comprising: positioning, by an operator, the plurality of robotic carts near the operating table based on a surgical procedure to be performed; adjusting, using a laser module provided with each of the plurality of robotic carts, a laser line associated with each of the plurality of robotic carts with respect to the frame of reference; transmitting, using a transmitter, encoded values of angles between the laser line associated with each of the plurality of robotic carts and the frame of reference, to a master controller; and calculating, using the master controller, the kinematics of the plurality of robotic carts with respect to the frame of reference, based on the encoded values of the angles.

Optionally, wherein the frame of reference may be a robotic cart having a camera or any other robotic cart out of the plurality of robotic carts.

Optionally, the laser lines associated with each of the plurality of robotic carts are kept perpendicular to plurality of robotic carts.

Optionally, the encoded values of angles between the laser line associated with each of the plurality of robotic carts and the frame of reference are transmitted to the master controller using either a wired or wireless communication.

Optionally, the kinematics of the plurality of robotic carts with respect to the frame of reference is independent of the type of operating room.

Optionally, the placement of the plurality of robotic carts in the operating room can be done as per the requirements of the surgical procedure to be performed.

Optionally, the maximum rotation for each of the plurality of robotic carts is within a range of ±160°.

Optionally, the laser module provided with each of the plurality of robotic carts is kept at an inclination to provide improved visualization.

Optionally, the length of the laser line can be up to 2 m.

Optionally, the laser module provided with each of the plurality of robotic carts is a class C laser module compatible with the medical standards.

Optionally, the kinematics of the plurality of robotic carts with respect to a frame of reference is obtained by visualization.

Optionally, each of the laser modules comprises of a knob gear, a laser gear, an encoder gear, an encoder, and a stopper.

Optionally, the laser module has a base cover and a top cover.

Optionally, a gear ratio of 3:1 is provided between the knob gear and the laser gear.

Optionally, a gear ratio of 1:1 is provided between the laser gear and the encoder gear.

Other embodiments, systems, methods, apparatus aspects, and features of the invention will become apparent to those skilled in the art from the following detailed description, the accompanying drawings, and the appended claims. It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of the disclosure, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to the scale. Wherever possible, like elements have been indicated by identical numbers.

Embodiments of the present disclosure will now be described, by way of example only, with reference to the following diagrams wherein:

FIG. 1 illustrates an example implementation of a multi arm teleoperated surgical system which can be used with one or more features in accordance with an embodiment of the disclosure;

FIG. 2 illustrates a five-arm configuration of robotic carts arranged around an operating table in a multi-arm teleoperated surgical system in accordance with an embodiment of the disclosure;

FIG. 3 illustrates an implementation of system for obtaining kinematics of a robotic cart in a multi-arm robotic surgical system in accordance with an embodiment of the disclosure;

FIGS. 4(a), 4(b), and 4(c) illustrate the laser module in accordance with an embodiment of the disclosure; and

FIG. 5 illustrates a flow chart with steps of a method for calculating the kinematics of the plurality of robotic carts with respect to a frame of reference in accordance with an embodiment of the disclosure.

### DETAILED DESCRIPTION OF THE DISCLOSURE

For the purpose of promoting an understanding of the principles of the disclosure, reference will now be made to the embodiment illustrated in the drawings and specific language will be used to describe the same. It will nevertheless be understood that no limitation of the scope of the disclosure is thereby intended, such alterations and further modifications in the illustrated system, and such further applications of the principles of the disclosure as illustrated therein being contemplated as would normally occur to one skilled in the art to which the disclosure relates.

It will be understood by those skilled in the art that the foregoing general description and the following detailed description are exemplary and explanatory of the disclosure and are not intended to be restrictive thereof. Throughout the patent specification, a convention employed is that in the appended drawings, like numerals denote like components.

Reference throughout this specification to “an embodiment”, “another embodiment”, “an implementation”, “another implementation” or similar language means that a particular feature, structure, or characteristic described in connection with the embodiment is included in at least one embodiment of the present disclosure. Thus, appearances of the phrase “in an embodiment”, “in another embodiment”, “in one implementation”, “in another implementation”, and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures proceeded by “comprises . . . a” does not, without more constraints, preclude the existence of other devices or other sub-systems or other elements or other structures or additional devices or additional sub-systems or additional elements or additional structures.

Unless otherwise defined, all technical and scientific terms used herein have the same meaning as commonly understood by one of ordinary skill in the art to which this disclosure belongs. The device, system, and examples provided herein are illustrative only and not intended to be limiting.

The terms “a” and “an” herein do not denote a limitation of quantity, but rather denote the presence of at least one of the referenced items. Further, the term sterile barrier and sterile adapter denotes the same meaning and may be used interchangeably throughout the description.

Embodiments of the disclosure will be described below in detail with reference to the accompanying drawings.

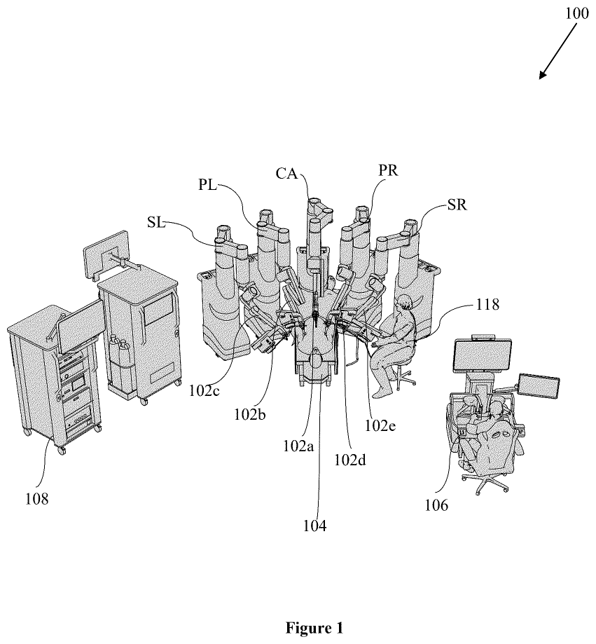

FIG. 1 illustrates an example implementation of a multi arm teleoperated surgical system which can be used with one or more features in accordance with an embodiment of the disclosure. Specifically, FIG. 1 illustrates the multi arm teleoperated surgical system (100) having five robotic arms (102a), (102b), (102c), (102d), (102e), mounted on five robotic arm carts around an operating table (104). The five-robotic arms (102a), (102b), (102c), (102d), (102e), as depicted in FIG. 1, are for illustration purposes and the number of robotic arms may vary depending upon the type of surgery. The exemplary five robotic arms (102a), (102b), (102c), (102d), (102e), are arranged along the operating table (104) and may be arranged in different manner but not limited to the robotic arms (102a), (102b), (102c), (102d), (102e), arranged along the operating table (104). The robotic arms (102a), (102b), (102c), (102d), (102e), may be separately mounted on the five robotic arm carts or the robotic arms (102a), (102b), (102c), (102d), (102e), mechanically and/or operationally connected with each other or the robotic arms (102a), (102b), (102c), (102d), (102e), connected to a central body (not shown) such that the robotic arms (102a), (102b), (102c), (102d), (102e), branch out of a central body (not shown). Further, the multi arm teleoperated surgical system (100) includes a master controller (106), a vision cart (108), and a surgical instrument and accessory table.

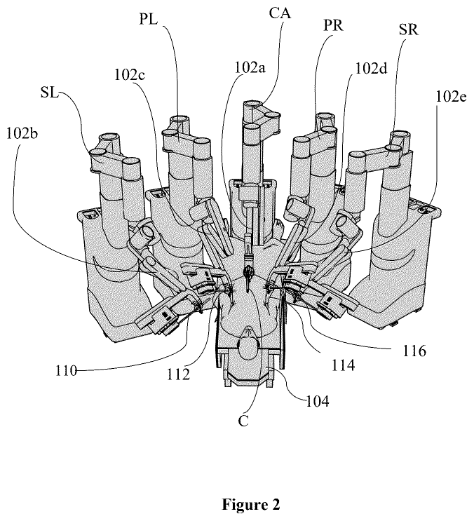

FIG. 2 illustrates a five-arm configuration of robotic carts arranged around an operating table in a multi-arm teleoperated surgical system in accordance with an embodiment of the disclosure. According to an embodiment, the patient side arm carts are indicated as camera arm cart (CA), primary right robotic arm cart (PR), secondary right robotic arm cart (SR), primary left robotic arm cart (PL), and secondary left robotic arm cart (SL). The right and left position of the patient side arm cart carts (PSAC) is named with respect to the surgeon's endoscopic view and not the physical placement of the carts. This is only for identification purposes. An endoscopic camera (C) is coupled to the robotic arm (102a) attached to the camera arm cart (CA). In robotic surgeries, sometimes a surgeon needs to hold a tissue or organ, while performing suturing, clipping, cutting, sealing, and coagulating etc. Then, one robotic arm out of the remaining robotic arms (102b, 102c, 102d, 102c) can be utilized to hold the above-mentioned tissue or organ. Two of the other remaining robotic arms (102b, 102c, 102d, 102c) can be used for other surgical actions. Each of the plurality of surgical instruments (110, 112, 114, 116) is detachably coupled to a robotic arm out of the remaining robotic arms (102b, 102c, 102d, 102e), which in turn is connected to a patient side arm cart out of patient side arm carts (SL, PL, PR, SR).

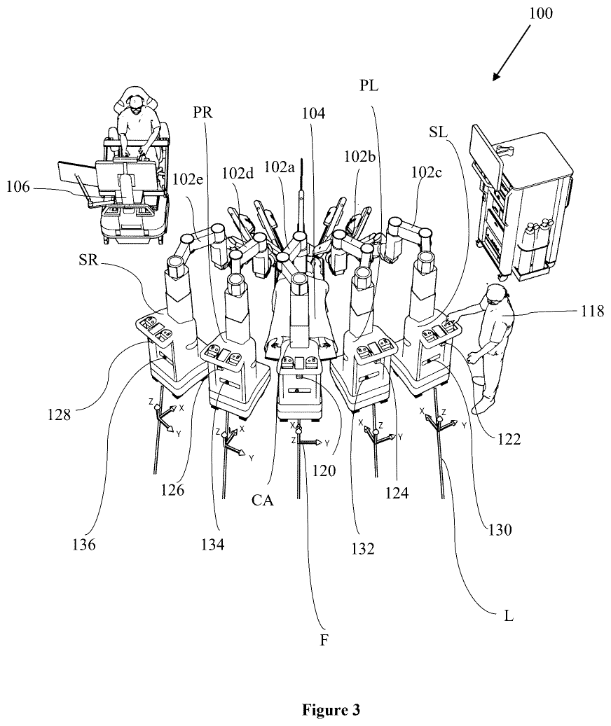

FIG. 3 illustrates an implementation of a system for obtaining kinematics of a robotic cart in a multi-arm robotic surgical system in accordance with an embodiment of the disclosure. Each of the plurality of robotic carts (SL, PL, PR, SR) is provided with a laser module (122, 124, 126, 128). The laser module (122, 124, 126, 128) is configured to generate a laser line (L) associated with each of the plurality of robotic carts (SL, PL, PR, SR). Each of the plurality of robotic carts (SL, PL, PR, SR) has an associated frame of orientation. The frame of reference associated with the robotic cart (CA) having a camera (C), is indicated by (F). A transmitter (130, 132, 134, 136) is coupled to each of the plurality of robotic carts (SL, PL, PR, SR). The transmitter (130, 132, 134, 136) is configured to encode values of angles between the generated laser line (L) associated with each of the plurality of robotic carts (SL, PL, PR, SR) and the frame of reference (F). A master controller (106) is operatively coupled to the plurality of robotic carts (SL, PL, PR, SR). The master controller (106) is configured to calculate the kinematics of the plurality of robotic carts (SL, PL, PR, SR) with respect to the frame of reference (F), based on the encoded values of the angles. The frame of reference (F) may be a robotic cart (CA) having a camera (C) or any other robotic cart out of the plurality of robotic carts (SL, PL, PR, SR).

The laser lines (L) associated with each of the plurality of robotic carts (SL, PL, PR, SR) are kept perpendicular to plurality of robotic carts (SL, PL, PR, SR). The encoded values of angles between the laser line (L) associated with each of the plurality of robotic carts (SL, PL, PR, SR) and the frame of reference (F) are transmitted to the master controller (106) using either a wired or wireless communication. The kinematics of the plurality of robotic carts (SL, PL, PR, SR) with respect to the frame of reference (F) is independent of the type of operating room. The placement of the plurality of robotic carts (SL, PL, PR, SR) in the operating room can be done as per the requirements of the surgical procedure to be performed.

The maximum rotation for each of the plurality of robotic carts (SL, PL, PR, SR) is within a range of ±160°. The laser module (122, 124, 126, 128) provided with each of the plurality of robotic carts (SL, PL, PR, SR) is kept at an inclination to provide improved visualization. The length of the laser line (L) can be up to 2 m. The laser module (122, 124, 126, 128) provided with each of the plurality of robotic carts (SL, PL, PR, SR) is a class C laser module compatible with the medical standards. The kinematics of the plurality of robotic carts (SL, PL, PR, SR) with respect to a frame of reference (F) is obtained by visualization. Each of the laser modules (122, 124, 126, 128) comprises of a knob gear (138), a laser gear (140), an encoder gear (142), an encoder (144), and a stopper (146). The laser module (122, 124, 126, 128) has a base cover (148) and a top cover (150). The base cover (148) holds all the components in a fixed place. The top cover (150) keeps the laser module (122, 124, 126, 128) enclosed and helps in attaching the laser module to the plurality of robotic carts (SL, PL, PR, SR). The stopper (146) is used to keep the laser gear (140), the knob gear (138), and the encoder gear (142) within a permissible limit. The stopper (146) also helps in stopping the rotation of laser gear beyond ±120°.

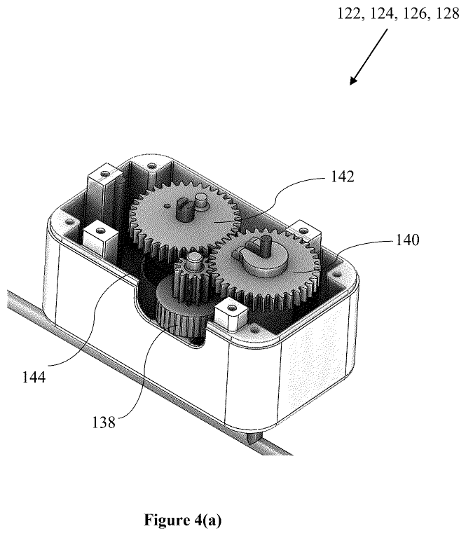

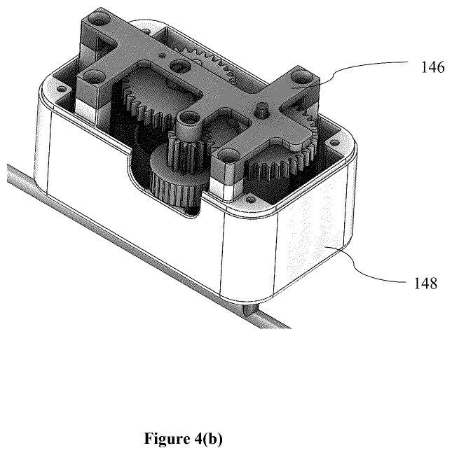

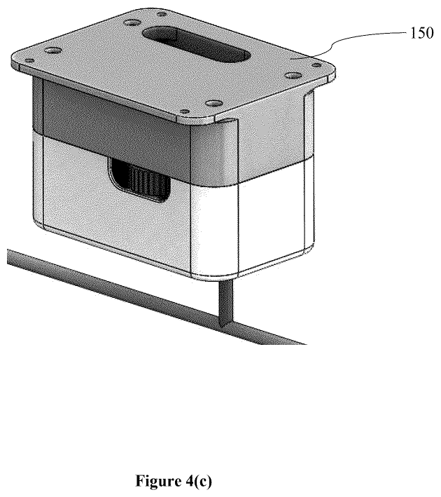

FIGS. 4(a), 4(b), and 4(c) illustrate a laser module for registration of a robotic cart in a multi-arm robotic surgical system. As the operator (118) switches on the power of the laser module, a laser line (L) associated with each of the plurality of robotic carts (SL, PL, PR, SR) is generated. For performing surgery, each of the plurality of robotic carts (SL, PL, PR, SR) must be aligned with a frame of reference (F) as shown in FIG. 3. This will help in obtaining the kinematics and orientation of each of the plurality of robotic carts (SL, PL, PR, SR) with respect to frame F. The frame of reference can be chosen as frame associated with the camera cart (CA). To align the carts, the operator (118) moves the knob gear (142). The movement of the knob gear (142) will move the laser gear (144). A gear ratio of 3:1 is provided between the knob gear (138) and the laser gear (140). This gear ratio of 3:1 provides a fine movement of the laser line (L) by giving a reduction of 3:1, so that even when the knob gear (138) is moved rapidly, a precise rotation of the laser line (L) is obtained. Also, a gear ratio of 1:1 is provided between the laser gear (140) and the encoder gear (142).

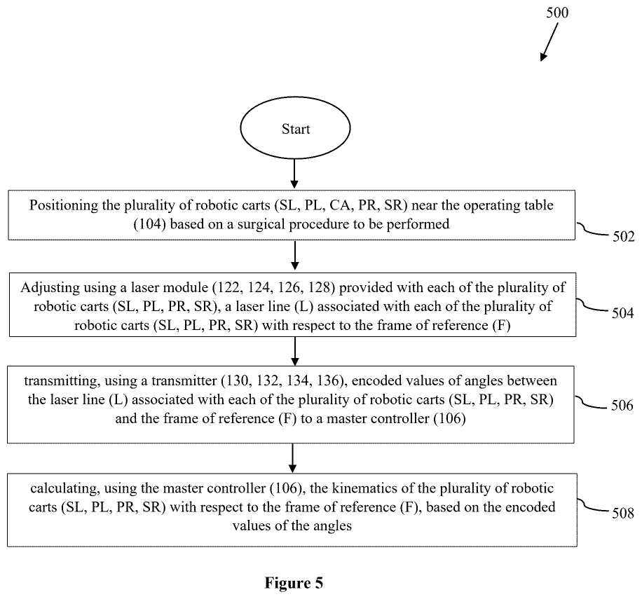

FIG. 5 illustrates a flow chart with steps of a method (500) for obtaining kinematics of a plurality of robotic carts (SL, PL, PR, SR) with respect to a frame of reference (F) in a multi-arm robotic surgical system (100) in accordance with an embodiment of the disclosure. At steps (502), the operator (118) positions the plurality of robotic carts (SL, PL, CA, PR, SR) near the operating table (104) based on a surgical procedure to be performed. At step (504), the operator (118) adjusts a laser line (L) associated with each of the plurality of robotic carts (SL, PL, PR, SR) with respect to the frame of reference (F), using a laser module (122, 124, 126, 128) provided with each of the plurality of robotic carts (SL, PL, PR, SR). A transmitter (130, 132, 134, 136) is provided with each of the plurality of robotic carts (SL, PL, PR, SR). At step (506), the transmitter (130, 132, 134, 136) encodes the values of angles between the laser line (L) associated with each of the plurality of robotic carts (SL, PL, PR, SR) and the frame of reference (F). The transmitter (130, 132, 134, 136) transmits these encoded values to a master controller (106) shown in FIG. 3. At step (508), the master controller (106) calculates the kinematics of the plurality of robotic carts (SL, PL, PR, SR) with respect to the frame of reference (F), based on the encoded values of the angles.

The multi-arm robotic surgical system (100) for obtaining kinematics of a plurality of robotic carts (SL, PL, PR, SR) with respect to a frame of reference (F) of the present disclosure is advantageous, as it can be effectively used in indoor environment like the operating room. Also, the present system is compliant with the medical regulations. Further, the system of the present disclosure does not impose any space restrictions and does not vary based on the type of operating room. Thus, in emergency conditions, the patient can be shifted from one operating room to another. Another advantage is that the system of the present disclosure is modular and thus, the surgeon's hand movement can be effectively mapped with the instrument tip.

The foregoing description of exemplary embodiments of the present disclosure has been presented for purposes of illustration and description. They are not intended to be exhaustive or to limit the disclosure to the precise forms disclosed, and obviously many modifications and variations are possible in light of the above teaching. The exemplary embodiment was chosen and described in order to best explain the principles of the disclosure and its practical application, to thereby enable others skilled in the art to best utilize the disclosure and various embodiments with various modifications as are suited to the particular use contemplated. It is understood that various omissions, substitutions of equivalents are contemplated as circumstance may suggest or render expedient but is intended to cover the application or implementation without departing from the spirit or scope of the claims of the present disclosure.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

While specific language has been used to describe the disclosure, any limitations arising on account of the same are not intended. As would be apparent to a person in the art, various working modifications may be made to the apparatus in order to implement the inventive concept as taught herein.

List of reference numerals:

| Sr. No. | Component | Reference Numeral(s) |
|---|---|---|
| 1 | Multi-arm robotic | 100 |
| | surgical system | |
| 2 | Robotic cart | SL, PL, CA, PR, SR |
| 3 | Frame of reference | F |
| 4 | Robotic arms | 102a, 102b, 102c, 102d, 102e |
| 5 | Endoscopic camera | C |
| 6 | Surgical instruments | 110, 112, 114, 116 |
| 7 | Operating table | 104 |
| 8 | Vision cart | 108 |
| 9 | Operator | 118 |
| 10 | Laser module | 122, 124, 126, 128 |
| 11 | Laser Line | L |
| 12 | Transmitter | 130, 132, 134, 136 |
| 13 | Master controller | 106 |
| 14 | Knob gear | 138 |
| 15 | Laser gear | 140 |
| 16 | Encoder gear | 142 |
| 17 | Encoder | 144 |
| 18 | Stopper | 146 |
| 19 | Base cover | 148 |
| 20 | Top cover | 150 |

1. A multi-arm robotic surgical system for obtaining kinematics of a plurality of robotic carts with respect to a frame of reference comprising a plurality of robotic arms each mounted on one of a plurality of robotic carts, an endoscopic camera coupled to a robotic arm out of the plurality of robotic arms, a plurality of surgical instruments each detachably coupled to a distal end of a robotic arm out of the remaining robotic arms, an operating table, and a patient lying on the operating table, whereby the plurality of robotic carts are arranged along the operating table, the system comprising:
a laser module provided with each of the plurality of robotic carts, the laser module configured to generate a laser line associated with each of the plurality of robotic carts;
a transmitter coupled to each of the plurality of robotic carts, the transmitter configured to encode values of angles between the generated laser line associated with each of the plurality of robotic carts and the frame of reference; and
a master controller operatively coupled to the plurality of robotic carts, the master controller configured to calculate the kinematics of the plurality of robotic carts with respect to the frame of reference, based on the encoded values of the angles.

2. The system as claimed in claim 1, wherein the frame of reference may be a robotic cart having a camera or any other robotic cart out of the plurality of robotic carts.

3. The system as claimed in claim 1, wherein the laser lines associated with each of the plurality of robotic carts are kept perpendicular to plurality of robotic carts.

4. The system as claimed in claim 1, wherein the encoded values of angles between the laser line associated with each of the plurality of robotic carts and the frame of reference are transmitted to the master controller using either a wired or wireless communication.

5. The system as claimed in claim 1, wherein the kinematics of the plurality of robotic carts with respect to the frame of reference is independent of the type of operating room.

6. The system as claimed in claim 1, wherein the placement of the plurality of robotic carts in the operating room can be done as per the requirements of the surgical procedure to be performed.

7. The system as claimed in claim 1, wherein the maximum rotation for each of the plurality of robotic carts is within a range of ±160°.

8. The system as claimed in claim 1, wherein the laser module provided with each of the plurality of robotic carts is kept at an inclination to provide improved visualization.

9. The system as claimed in claim 1, wherein the length of the laser line can be up to 2 m.

10. The system as claimed in claim 1, wherein the laser module provided with each of the plurality of robotic carts is a class C laser module compatible with the medical standards.

11. The system as claimed in claim 1, wherein the kinematics of the plurality of robotic carts with respect to a frame of reference is obtained by visualization.

12. The system as claimed in claim 1, wherein each of the laser module comprises of a knob gear, a laser gear, an encoder gear, an encoder, and a stopper.

13. The system as claimed in claim 12, wherein the laser module has a base cover and a top cover.

14. The system as claimed in claim 12, wherein a gear ratio of 3:1 is provided between the knob gear and the laser gear.

15. The system as claimed in claim 12, wherein a gear ratio of 1:1 is provided between the laser gear and the encoder gear.

16. A method for obtaining kinematics of a plurality of robotic carts with respect to a frame of reference in a multi-arm robotic surgical system comprising a plurality of robotic arms each mounted on one of a plurality of robotic carts, an endoscopic camera coupled to a robotic arm out of the plurality of robotic arms, a plurality of surgical instruments each detachably coupled to a distal end of a robotic arm out of the remaining robotic arms, an operating table, and a patient lying on the operating table, whereby the plurality of robotic carts are arranged along the operating table, the method comprising:
positioning, by an operator, the plurality of robotic carts near the operating table based on a surgical procedure to be performed;
adjusting, using a laser module provided with each of the plurality of robotic carts, a laser line associated with each of the plurality of robotic carts with respect to the frame of reference;
transmitting, using a transmitter, encoded values of angles between the laser line associated with each of the plurality of robotic carts and the frame of reference, to a master controller; and
calculating, using the master controller, the kinematics of the plurality of robotic carts with respect to the frame of reference, based on the encoded values of the angles.
